(19) United States
(12) Patent Application Publication
(10) Pub. No.: US 2021/0114990 A1
(43) Pub. Date: Apr. 22, 2021

(54) DISUBSTITUTED 3-PYRAZOLE CARBOXYLATES AND A PROCESS FOR THEIR PREPARATION VIA ACYLATION OF ENOLATES

(71) Applicant: BAYER AKTIENGESELLSCHAFT, Leverkusen (DE)

(72) Inventors: Sergii PAZENOK, Leichlingen (DE); Anton LISHCHYNSKYI, Solingen (DE); Julia Johanna HAHN, Duesseldorf (DE); Frank MEMMEL, Guntersblum (DE); Mark James FORD, Niedernhausen (DE)

(21) Appl. No.: 17/253,001

(22) PCT Filed: Jun. 14, 2019
(86) PCT No.: PCT/EP2019/065642

§ 371 Date: Dec. 16, 2020

(30) Foreign Application Priority Data
Jun. 18, 2018 (EP) 18178284.8

(51) Int. Cl. C07D 231/18

(52) U.S. Cl. CPC C07D 231/18

(57) ABSTRACT

The present invention relates to disubstituted 3-pyrazole carboxylates of the formula (I) and a process for their preparation

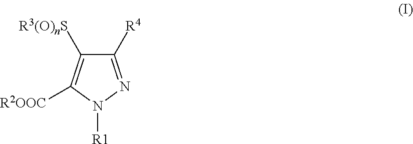

wherein R¹, R², R³, R⁴ and n are defined as above.

The present invention relates to disubstituted 3-pyrazole carboxylates and a novel process for their preparation. It is known from WO 2012/126766 that N-Alkyl-3-haloalkyl-4-(methylsulfinyl)-5-pyrazoles carboxylates are important precursors for the synthesis of pyrazole carboxyamides which possess strong insecticidal activity. The chemical synthesis of a pyrazole with C₂F₅-group in position 3 and SMe-group in position 4 was described in WO 2012/126766. This synthesis however requires multi step transformations with moderate yield and tedious isolation and purification.

Synthesis of mono halogenalkyl substituted pyrazole carboxylates utilizing an acylation reaction has been already documented in WO 2018/054807 and WO 2009/106230.

In the light of the prior art described above, it is an object of the present invention to provide a process that does not have the aforementioned disadvantages and hence gives a route to disubstituted 5-pyrazole carboxylates derivatives in high yields.

The object described above was achieved by a process for the preparation of disubstituted 3-pyrazole carboxylates of the formula (I)

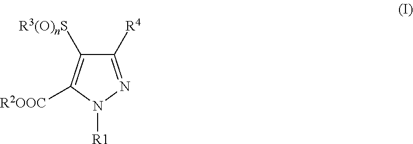

wherein
R¹ is selected from H, (C₁-C₆)alkyl, (C₃-C₈)cycloalkyl, phenyl or 2-pyridyl,
R² is selected from H, (C₁-C₁₂)alkyl or (C₃-C₈)cycloalkyl,
R³ is selected from (C₁-C₁₂)alkyl, (C₁-C₃)haloalkyl, (C₃-C₈)cycloalkyl, (C₆-C₁₂)aryl, (C₁-C₃)alkyl(C₆-C₁₂)aryl and (C₆-C₁₂)aryl(C₁-C₆)alkyl,
R⁴ is selected from (C₁-C₆)haloalkyl and (C₁-C₃)haloalkoxy(C₁-C₆)haloalkyl and
n is 0, 1 or 2,

comprising a step (A), wherein acid derivatives of the formula (II),

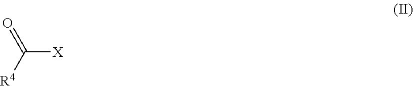

in which

R⁴ is defined as above and

X is selected from F, Cl, Br or —OC(O)R⁴

are, in the presence of a base, reacted with enolates of the formula (III),

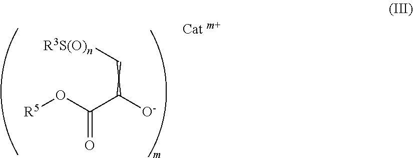

in which

R⁵ is selected from (C₁-C₁₂)alkyl, (C₆-C₁₂)aryl(C₁-C₆)alkyl, (C₆-C₁₂)aryl or (C₃-C₈)cycloalkyl,

n and R³ are defined as above,

m is 1 or 2 and

Cat^{m+} is selected from alkaline metal cations (with m=1), alkaline earth metal cations (with m=2), organic ammonium cations (with m=1) or organic phosphonium cations (with m=1)

to form compounds of formula (IV)

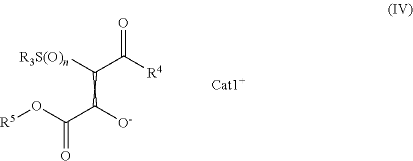

in which

n, R³, R⁴ and R⁵ are defined as above and

Cat¹⁺ is selected from alkaline metal cations, N-methylimidazolium cation, N-butylimidazolium cation, pyridinium cation, (C₁-C₄)alkylpyridinium cations, dimethylaminopyridinium cation, 4-aza-1-azoniabicyclo[2.2.2]octane cation, 1-methyl-2,3,4,6,7,8,9,10-octahydropyrimido[1,2-a]azepin-1-ium cation or organic ammonium cations of the general formula (R⁶)₃NH⁺,

wherein

R⁶ are each independently selected from (C₁-C₆)alkyl or (C₃-C₈)cycloalkyl

and further comprising a step (B), wherein cyclization with an hydrazine of the formula (V)

NH₂NHR¹ (V)

takes place to form the compounds of formula (I).

Preferred is a process according to the invention, where the radicals in formula (I), (II), (III), (IV) and (V) are defined as follows:
R¹ is selected from H, (C₁-C₆)alkyl or (C₃-C₈)cycloalkyl, phenyl or 2-pyridyl,
R² is selected from H, (C₁-C₆)alkyl or (C₃-C₆)cycloalkyl,
R³ is selected from (C₁-C₆)alkyl, (C₁-C₃)haloalkyl, (C₃-C₆)cycloalkyl, (C₆-C₉)aryl, (C₁-C₃)alkyl(C₆-C₉)aryl and (C₆-C₉)aryl(C₁-C₃)alkyl,
R⁴ is selected from (C₁-C₆)haloalkyl and (C₁-C₃)haloalkoxy(C₁-C₆)haloalkyl, wherein the halogen is selected from fluoro and/or chloro,
R⁵ is selected from (C₁-C₆)alkyl or (C₃-C₆)cycloalkyl,
n is 0, 1 or 2,
m is 1,
Cat^{m+} is selected from alkaline metal cations, preferably from Li⁺, Na⁺, K⁺ and Cs⁺, organic ammonium cations, preferably (R⁷)₄N⁺ or organic phosphonium cations, preferably (phenyl)₄P⁺, wherein
R⁷ are each independently selected from (C₁-C₆)alkyl or (C₆-C₁₂)aryl and
X is selected from F, Cl, Br or —OC(O)R⁴.

More preferred is a process according to the invention, where the radicals in formula (I), (II), (III), (IV) and (V) are defined as follows:
R¹ is selected from H or (C₁-C₆)alkyl,
R² is selected from H or (C₁-C₆)alkyl,
R³ is selected from (C₆-C₉)aryl or (C₁-C₆)alkyl,
R⁴ is selected from difluoromethyl, trifluoromethyl, chlorofluoromethyl, dichlorofluoromethyl, chlorodifluoromethyl, 1-fluoroethyl, 2-fluoroethyl, 2,2-difluoroethyl, 2,2,2-trifluoroethyl, 2-chloro-2-fluoroethyl, 2-difluoroethyl, 2,2-dichloro-2-fluoroethyl, 2,2,2-trichloroethyl, 1,2,2,2-tetrafluoroethyl (CF₃CFH), pentafluoroethyl, heptafluoropropyl, trifluoromethoxyfluoromethyl (CF₃OCFH)— and 1,1,1-trifluoroprop-2-yl;
R⁵ is selected from (C₁₋₆)alkyl,
n is 0, 1 or 2,
m is 1,
Cat^{m+} is selected from Li⁺, Na⁺, K⁺ and Cs⁺ and (R⁷)₄N⁺, wherein
R⁷ are each independently selected from (C₁-C₂)alkyl and
X is independently selected from F, Cl, Br or —OC(O)R⁴.

Even more preferred is a process according to the invention, where the radicals in formula (I), (II), (III), (IV) and (V) are defined as follows:
R¹ is selected from H, methyl, ethyl or iso-propyl,
R² is selected from H, methyl or ethyl,
R³ is selected from methyl, ethyl or phenyl,
R⁴ is selected from difluoromethyl, trifluoromethyl, pentafluoroethyl or heptafluoropropyl,
R⁵ is selected from methyl, ethyl, propyl or iso-propyl,
n is 2,
m is 1,
Cat^{m+} is selected from Li⁺, Na⁺, K⁺ and Cs⁺ and
X is F, Cl or —OC(O)R⁴.

Most preferred is a process according to the invention, where the radicals in formula (I), (II), (III), (IV) and (V) are defined as follows:
R¹ is selected from H or methyl,
R² is selected from H, methyl or ethyl,
R³ is methyl,
R⁴ is selected from trifluoromethyl, pentafluoroethyl or heptafluoropropyl,
R⁵ is selected from methyl or ethyl,
n is 2,
m is 1,
Cat^{m+} is selected from Na⁺ and K⁺ and
X is F, Cl or —OC(O)R⁴.

In a particularly preferred embodiment of the present invention n is 2 for the compounds of the general formula (I), (III) and (IV).

In a preferred embodiment of the invention is the process carried out in the presence of one or more suitable solvents. Suitable solvents will be specified below for the respective process steps.

Surprisingly, the pyrazoles of the formula (I) can be prepared under the inventive conditions with good yields and in high purity, which means that the process according to the invention overcomes the abovementioned disadvantages of the preparation processes previously described in the prior art.

An object of the present invention are also disubstituted 3-pyrazole carboxylates of the formula (I),

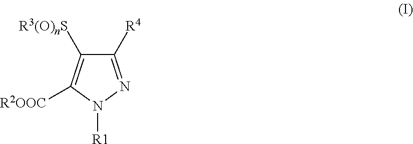

wherein
R1 is selected from H, (C1-C6)alkyl, (C3-C8)cycloalkyl, phenyl or 2-pyridyl,
R2 is selected from H, (C1-C12)alkyl or (C3-C8)cycloalkyl,
R³ is selected from (C₁-C₁₂)alkyl, (C₁-C₃)haloalkyl, (C₃-C₈)cycloalkyl, (C₆-C₁₂)aryl, (C₁-C₃)alkyl(C₆-C₁₂)aryl and (C₆-C₁₂)aryl(C₁-C₆)alkyl,
R⁴ is selected from (C₁-C₆)haloalkyl and (C₁-C₃)haloalkoxy(C₁-C₆)haloalkyl and
n is 2.

Preferred are disubstituted 3-pyrazole carboxylates of formula (I), wherein
R¹ is selected from H, (C₁-C₆)alkyl, (C₃-C₈)cycloalkyl, phenyl or 2-pyridyl,
R² is selected from H, (C₁-C₆)alkyl or (C₃-C₆)cycloalkyl,
R³ is selected from (C₁-C₆)alkyl, (C₁-C₃)haloalkyl, (C₃-C₆)cycloalkyl, (C₆-C₉)aryl, (C₁-C₃)alkyl(C₆-C₉)aryl and (C₆-C₉)aryl(C₁-C₃)alkyl,
R⁴ is selected from (C₁-C₆)haloalkyl and (C₁-C₃)haloalkoxy(C₁-C₆)haloalkyl, wherein the halogen is selected from fluoro and/or chloro and
n is 2.

More preferred are disubstituted 3-pyrazole carboxylates of formula (I), wherein
R¹ is selected from H or (C₁-C₆)alkyl,
R² is selected from H or (C₁-C₆)alkyl,
R³ is selected from (C₆-C₉)aryl or (C₁-C₆)alkyl,
R⁴ is selected from difluoromethyl, trifluoromethyl, chlorofluoromethyl, dichlorofluoromethyl, chlorodifluoromethyl, 1-fluoroethyl, 2-fluoroethyl, 2,2-difluoroethyl, 2,2,2-trifluoroethyl, 2-chloro-2-fluoroethyl, 2-difluoroethyl, 2,2-dichloro-2-fluoroethyl, 2,2,2-trichloroethyl, 1,2,2,2-tetrafluoroethyl (CF₃CFH), pentafluoroethyl, heptafluoropropyl, trifluoromethoxyfluoromethyl (CF₃OCFH)— and 1,1,1-trifluoroprop-2-yl and
n is 2.

Even more preferred are disubstituted 3-pyrazole carboxylates of formula (I), wherein
R¹ is selected from H, methyl, ethyl or iso-propyl,
R² is selected from H, methyl or ethyl,
R³ is selected from methyl, ethyl or phenyl,
R⁴ is selected from difluoromethyl, trifluoromethyl, pentafluoroethyl or heptafluoropropyl and
n is 2.

Most preferred are disubstituted 3-pyrazole carboxylates of formula (I), wherein
R¹ is selected from H or methyl,
R² is selected from H, methyl or ethyl,
R³ is methyl,
R⁴ is selected from trifluoromethyl, pentafluoroethyl or heptafluoropropyl and
n is 2.

A further object of the present invention are intermediates of the general formula (IV)

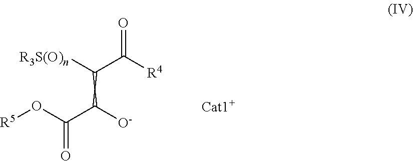

in which

n, R⁴ and R⁵ are defined as above,

R³ is selected from (C₁-C₁₂)alkyl, (C₁-C₃)haloalkyl or (C₃-C₈)cycloalkyl and

Cat1⁺ is selected from alkaline metal cations, N-methylimidazolium cation, N-butylimidazolium cation, pyridinium cation, (C₁-C₄)alkylpyridinium cations, dimethylaminopyridinium cation, 4-aza-1-azoniabicyclo[2.2.2]octane cation, 1-methyl-2,3,4,6,7,8,9,10-octahydropyrimido[1,2-a]azepin-1-ium cation or organic ammonium cations of the general formula (R⁶)₃NH⁺,

wherein

R⁶ are each independently selected from (C₁-C₆)alkyl or (C₃-C₈)cycloalkyl.

Preferred are intermediates of formula (IV), wherein Cat1⁺ of formula (IV) is selected from organic ammonium cations of the general formula (R⁶)₃NH⁺,

wherein

R⁶ are each independently selected from (C₁-C₄)alkyl or (C₃-C₆)cycloalkyl.

More preferred are intermediates of formula (IV), wherein Cat1⁺ of formula (IV) is selected from N(iPr)₂(Et)H⁺, N(Me)₃H⁺, (Me)₂N(cyclohexyl)H⁺, N(Et)₃H⁺ or N(Bu)₃H⁺.

Also preferred are intermediates of formula (IV), wherein R³ is selected from (C₁-C₆)alkyl or (C₁-C₃)haloalkyl, more preferred from (C₁-C₆)alkyl and even more preferred from ethyl or methyl and most preferred R³ is methyl.

### General Definitions

In the context of the present invention, the term “halogen” (Hal), unless defined differently, comprises those elements which are selected from the group comprising fluorine, chlorine, bromine and iodine, preferably fluorine, chlorine and bromine, more preferably fluorine and chlorine.

Alkyl groups in the context of the present invention, unless defined differently, are linear or branched saturated hydrocarbyl groups. The definition C₁-C₁₂-alkyl encompasses the widest range defined herein for an alkyl group. Specifically, this definition encompasses, for example, the meanings of methyl, ethyl, n-, isopropyl, n-, iso-, sec- and t-butyl, n-pentyl, n-hexyl, 1,3-dimethylbutyl, 3,3-dimethylbutyl, n-heptyl, n-nonyl, n-decyl, n-undecyl or n-dodecyl.

The term Alkoxy, either on its own or else in combination with further terms, for example haloalkoxy, is understood in the present case to mean an O-alkyl radical, where the term “alkyl” is as defined above.

Cycloalkyl groups in the context of the present invention are monocyclic, saturated hydrocarbyl groups having 3 to 8 and preferably 3 to 6 carbon ring members, for example (but not limited to) cyclopropyl, cyclopentyl and cyclohexyl. This definition also applies to cycloalkyl as part of a composite substituent, for example cycloalkylalkyl etc., unless defined elsewhere.

Aryl groups in the context of the present invention, unless defined differently, are aromatic hydrocarbyl groups. The definition C₆₋₁₂-aryl encompasses the widest range defined herein for an aryl group having 6 to 12 skeleton atoms. The aryl groups may be mono- or bicyclic. Specifically, this definition encompasses, for example, the meanings of phenyl, cycloheptatrienyl, cyclooctatetraenyl, naphthyl and anthracenyl.

Arylalkyl groups (aralkyl groups) in the context of the present invention, unless defined differently, are alkyl groups which are substituted by aryl groups. Specifically, this definition encompasses, for example, the meanings of benzyl and phenylethyl.

Alkylaryl groups (alkaryl groups) in the context of the present invention, unless defined differently, are aryl groups which are substituted by one or more alkyl groups, which may have 1 to 6 carbon atoms in the alkyl chain. Specifically, this definition encompasses, for example, the meanings of tolyl or 2,3-, 2,4-, 2,5-, 2,6-, 3,4- or 3,5-dimethylphenyl.

Halogen-substituted radicals, for example haloalkyl, are mono- or polyhalogenated, up to the maximum number of possible substituents. In the case of polyhalogenation, the halogen atoms may be identical or different. Unless stated otherwise, optionally substituted radicals may be mono- or polysubstituted, where the substituents in the case of polysubstitutions may be the same or different.

Haloalkyl groups in the context of the present invention are straight-chain or branched alkyl groups having 1 to 6 and preferably 1 to 3 carbon atoms (as specified above), where some or all of the hydrogen atoms in these groups may be replaced by halogen atoms as specified above, for example (but not limited to) C₁-C₃-haloalkyl such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro, 2-difluoroethyl, 2,2-dichloro-2-fluoroethyl, 2,2,2-trichloroethyl, pentafluoroethyl and 1,1,1-trifluoroprop-2-yl. This definition also applies to haloalkyl as part of a composite substituent, for example haloalkylalkoxy, haloalkoxyhaloalkyl, haloalkylaminoalkyl etc., unless defined elsewhere. Preference is given to alkyl groups substituted by one or more halogen atoms, for example trifluoromethyl (CF₃), difluoromethyl (CHF₂), CF₃CFH, CF₃CH₂, CF₂Cl CF₃CF₂, CF₃CCl₂.

The term intermediate used in the context of the present invention describes the substances which occur in the process according to the invention and are prepared for further chemical processing and are consumed or used therein in order to be converted to another substance. The intermediates can often be isolated and intermediately stored or are used without prior isolation in the subsequent reaction step. The term “intermediate” also encompasses the generally unstable and short-lived intermediates which occur transiently in multistage reactions (staged reactions) and to which local minima in the energy profile of the reaction can be assigned.

The inventive compounds may be present as mixtures of any different isomeric forms possible, especially of stereoisomers, for example E and Z isomers, threo and erythro isomers, and optical isomers, but if appropriate also of tautomers. Both the E and the Z isomers are disclosed and claimed, as are the threo and erythro isomers, and also the optical isomers, any mixtures of these isomers, and also the possible tautomeric forms.

### PROCESS DESCRIPTION

The process according to the invention is illustrated in Scheme 1, wherein X, R¹, R³, R⁴, R⁵, n, m, Cat^{m+}, y and Cat1^{y+}, are defined as above:

Scheme 1:

Step (A)

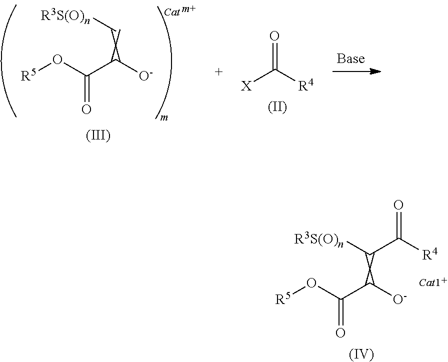

Step (B)

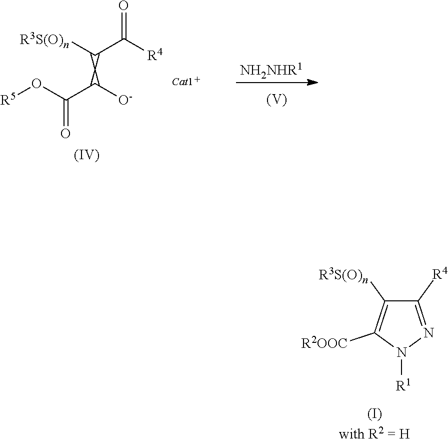

Step (A):

In step (A), acid derivatives of the formula (II) are first reacted, in the presence of a base, with compounds of the formula (III).

Preferred compounds of the general formula (II) used to introduce R⁴=trifluoromethyl, difluoromethyl or heptafluoropropyl are for example trifluoroacetylchloride, trifluoracetylfluoride, difluoracetylfluoride, difluoroacetylchloride, trifluoroacetylbromide, heptafluorobutyric anhydride, heptafluorobutanoyl fluoride, heptafluorobutanoyl chloride and heptafluorobutanoyl bromide.

It is also possible to generate the compounds of formula (II) in situ, for instance using trifluoroacetic acid, pivaloyl chloride and pyridine as described in WO 2003/051820.

For the introduction of R⁴=pentafluoroethyl it is preferred to use pentafluoropropionyl fluoride or pentafluoropropionic anhydride.

It is further preferred to use hexafluoropropenoxide as starting material for the introduction of R⁴=pentafluoroethyl. Hexafluoropropenoxide can form “in situ” pentafluoropropionyl fluoride as compound of the general formula (II) as generally described in Zhurnal Organicheskoi Khimii, vol. 24, N. 7. pp. 1559-1560, 1988.

The formation of pentafluoropropionyl fluoride from hexafluoropropenoxide can be effected in the presence of a base, preferably trialkylamines (R⁶)₃N, wherein R⁶ are each independently selected from (C₁-C₆)alkyl or (C₃-C₈)cycloalkyl, preferably (C₁-C₄)alkyl or (C₃-C₆)cycloalkyl, more preferably from methyl, ethyl, butyl, cyclohexyl (Cy) or iso-propyl. More preferable the base is selected from N(iPr)₂(Et), (Me)₂N(Cy), N(Me)₃, N(Et)₃ or N(Bu)₃ and most preferable from N(Et)₃ or N(Bu)₃. Most preferably the base used for step (A) is selected to be suitable to effect the formation of pentafluoropropionyl fluoride from hexafluoropropenoxide and no further base is added.

The formation of pentafluoropropionyl fluoride from hexafluoropropenoxide is preferably effected at temperatures between −80° C. to +100° C., more preferably at temperatures of −15° C. to +50° C., even more preferably at −5 to +30° C.

Compounds of formula (III) can be prepared from the cheap and available chemicals like methylalkylsulphones and oxalic acid esters according to Sokolov, M. P. et al; Journal of Organic Chemistry USSR (English Translation); vol. 22; (1986); p. 644-647. Preferred compounds of the formula (III) are sodium 3-methoxy-1-(methylsulfonyl)-3-oxoprop-1-en-2-olates, sodium 3-ethoxy-1-(methylsulfonyl)-3-oxoprop-1-en-2-olates, sodium 3-ethoxy-1-(phenylsulfonyl)-3-oxoprop-1-en-2-olates, potassium 3-methoxy-1-(methylsulfonyl)-3-oxoprop-1-en-2-olates and potassium 3-ethoxy-1-(methylsulfonyl)-3-oxoprop-1-en-2-olates.

The compounds of formula (III) can also be formed “in situ” from the compounds of formula (VI) or (VII) in the presence of a base. The compounds of formula (VI) and (VII) are tautomers and are both present in an equilibrium. For compounds of the formula (VI) and (VII) R³, R and n are defined as above. The base can be selected from alkali metal (C₁-C₄)alkoxides, for example LiOMe, NaOMe, NaOEt, NaOt-But, KOMe or KOt-Bu. For this step according to the invention preferably 1 to 5 mol, more preferred 1 to 2 mol and even more preferred 1 to 1.5 mol of the base are used.

The formation of compounds of formula (III) from compounds of formula (VI) and (VII) is preferably effected at temperatures between 0° C. to 40° C., more preferably at temperatures of 5° C. to 30° C., even more preferably at 20 to 30° C.

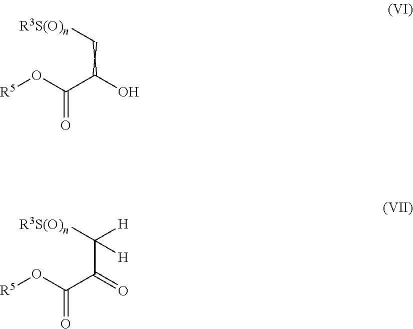

The step (A) according to the invention is preferably effected at temperatures of −80° C. to +100° C., more preferably at temperatures of −15° C. to +50° C., even more preferably at −5 to +30° C. and under standard pressure.

Step (A) takes place in the presence of a base. Preference is given to organic bases, such as trialkylamines (R⁶)₃N, wherein R⁶ are each independently selected from (C₁-C₆)alkyl or (C₃-C₈)cycloalkyl, preferably (C₁-C₄)alkyl or (C₃-C₆)cycloalkyl, more preferably from methyl, ethyl, butyl, cyclohexyl (Cy) or iso-propyl; pyridine, (C₁-C₄)alkylpyridines, preferably picolines; N-methylimidazole, N-butylimidazole, dimethylaminopyridine, 1,4-Diazabicyclo[2.2.2]octan (DABCO) and 1,8-diazabicyclo[5.4.0]undecene (DBU) or alkali metal hydroxides, for example lithium hydroxide, sodium hydroxide or potassium hydroxide; alkali metal carbonates, for example Na₂CO₃ or K₂CO₃; alkali metal (C₁-C₄)alkoxides, for example NaOMe, NaOEt, NaOt-But or KOt-But; or alkali metal fluorides, for example KF. Also mixtures of those bases could be used. Preferably the base is selected from trialkylamines (R⁶)₃N, more preferably from (Me)₂N(Cy), N(iPr)₂(Et), N(Me)₃, N(Et)₃ or N(Bu)₃ and even more preferably from N(Et)₃ or N(Bu)₃.

For step (A) according to the invention preferably 0.5 to 10 mol, more preferred 0.5 to 1.5 mol and even more preferred 1 to 1.25 mol of the base are used.

The reaction time is not critical and may, according to the batch size and temperature, be selected within a range between a few minutes and several hours.

For the process according to the invention preferably 0.5 to 2 mol, more preferred 1 to 1.5 mol and even more preferred 1 to 1.1 mol of the acid derivatives of the formula (II) are reacted with 1 mol compound of formula (III).

During step (A) compounds of the general formula (IV) are formed.

Cat1⁺ of formula (IV) is selected from alkaline metal cations, N-methylimidazolium cation, N-butylimidazolium cation, pyridinium cation, (C₁-C₄)alkylpyridinium cations, dimethylaminopyridinium cation, 4-aza-1-azoniabicyclo[2.2.2]octane cation, 1-methyl-2,3,4,6,7,8,9,10-octahydropyrimido[1,2-a]azepin-1-ium cation or organic ammonium cations of the general formula (R⁶)₃NH⁺,

wherein

R⁶ are each independently selected from (C₁-C₆)alkyl or (C₃-C₈)cycloalkyl.

Preferably Cat1⁺ of formula (IV) is selected from organic ammonium cations of the general formula (R⁶)₃NH⁺,

wherein

R⁶ are each independently selected from (C₁-C₆)alkyl or (C₃-C₈)cycloalkyl.

More preferably Cat1⁺ of formula (IV) is selected from ammonium cations of the general formula (R⁶)₃NH⁺,

wherein R⁶ are each independently selected from (C₁-C₄)alkyl or (C₃-C₆)cycloalkyl.

Even more preferably Cat1⁺ of formula (IV) is selected from N(iPr)₂(Et)H⁺, N(Me)₂(Cy)H⁺, N(Me)₃H⁺, N(Et)₃H⁺ or N(Bu)₃H⁺.

Most preferably Cat1⁺ of formula (IV) is selected from N(Et)₃H⁺ or N(Bu)₃H⁺.

Suitable solvents for step (A) are, for example, aliphatic, alicyclic or aromatic hydrocarbons, for example petroleum ether, n-hexane, n-heptane, cyclohexane, methylcyclohexane, benzene, toluene, xylene or decalin, and halogenated hydrocarbons, for example chlorobenzene, dichlorobenzene, dichloromethane, chloroform, tetrachloromethane, dichloroethane or trichloroethane, ethers such as diethyl ether, diisopropyl ether, methyl tert-butyl ether (MeOBu-t), methyl tert-amyl ether, dioxane, tetrahydrofuran (THF), 1,2-dimethoxyethane, 1,2-diethoxyethane or anisole, esters such as ethylacetate (EtOAc) or isopropylacetat, nitriles such as acetonitrile, propionitrile, n- or isobutyronitrile or benzonitrile; amides such as N,N-dimethylformamide, N,N-dimethylacetamide, N-methylformanilide, N-methylpyrrolidone or hexamethylphosphoramide; sulphoxides such as dimethyl sulphoxide or sulphones such as sulpholane. Particular preference is given, to THF, acetonitrile, MeOBu-t, dichloromethane, EtOAc, toluene, xylene, chlorobenzene, n-hexane, cyclohexane or methylcyclohexane, and very particular preference to toluene, dichloromethane, THF, MeOBu-t, acetonitrile or EtOAc.

The formed intermediates of the formula (IV) can be used in the cyclization step (B) without prior workup.

Alternatively, the intermediates can be isolated by suitable workup steps, characterized and optionally further purified.

The compounds of formula (IV) could also be transferred to compounds of formula (VIII) and (IX) by acidification according to Sokolov, M. P. et al; Zhurnal Organicheskoi Khimii, vol. 22, N. 4. pp. 721-724, 1986. The compounds of formula (VIII) and (IX) are tautomers and are both present in an equilibrium. For compounds of the formula (VIII) and (IX) R³, R⁴ and R⁵ are defined as above and n is 2.

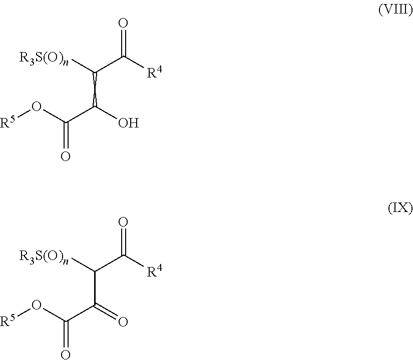

Step (B):

In the cyclization step (B) compound of formula (IV) are reacted with hydrazines of formula (V).

The reaction is effected at temperatures of −20° C. to +80° C., preferably at temperatures of +0° C. to +70° C., more preferably at +20 to +50° C. and under standard pressure. The reaction time is not critical and may, according to the batch size, be selected within a relatively wide range.

According to the invention preferably 1 mol to 2 mol, more preferably 1 to 1.5 mol of the hydrazine are used for the conversion 1 mol of the compound of formula (IV).

Preferably the cyclization step (B) is effected without changing the solvent after step (A).

Suitable solvents are, for example, aliphatic, alicyclic or aromatic hydrocarbons, for example petroleum ether, n-hexane, n-heptane, cyclohexane, methylcyclohexane, benzene, toluene, xylene or decalin, and halogenated hydrocarbons, for example chlorobenzene, dichlorobenzene, dichloromethane, chloroform, tetrachloromethane, dichloroethane or trichloroethane, ethers such as diethyl ether, diisopropyl ether, methyl tert-butyl ether (MeOBu-t), methyl tert-amyl ether, dioxane, tetrahydrofuran (THF), 1,2-dimethoxyethane, 1,2-diethoxyethane or anisole, alcohols such as methanol, ethanol, isopropanol or butanol, esters such as ethylacetate (EtOAc) or isopropylacetat, nitriles such as acetonitrile, propionitrile, n- or isobutyronitrile or benzonitrile; amides such as N,N-dimethylformamide, N,N-dimethylacetamide, N-methylformanilide, N-methylpyrrolidone or hexamethylphosphoramide, sulphoxides such as dimethyl sulphoxide or sulphones such as sulpholane. Particular preference is given to acetonitrile, THF, MeOBu-t, dichloromethane, EtOAc, toluene, xylene, chlorobenzene, n-hexane, cyclohexane or methylcyclohexane, and very particular preference to toluene, dichloromethane, THF, MeOBu-t, acetonitrile or EtOAc.

After the reaction has ended the compounds of the general formula (I) can be isolated and purified by suitable methods known to any person skilled in the art. For example, the solvents can be removed and the product can be isolated by filtration, or the product can be first washed with water, which will preferably be acidified with an acid, preferably with HCl or H₂SO₄, and extracted, the organic phase can be separated and the solvent can be removed under reduced pressure.

The compounds of the formula (I) where R²═H can be converted in a further step (C) to pyrazole acids ester of the formula (I) with R²═(C₁-C₁₂)alkyl or (C₃-C₈)cycloalkyl according to state of the art procedures as for example described by Xiong, Li et al., Journal of Agricultural and Food Chemistry, 65(5), 1021-1029; 2017 (see for example Scheme 2).

Scheme 2, Step (C):

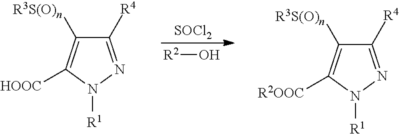

The process of the present invention preferably consists of steps A and B and optionally step C and also optionally the “in situ” formation of compound (II) from precursors as mentioned above.

### EXAMPLES

The invention is illustrated by but not limited to the following examples:

### Example 1

### Step (A)

### 1-Ethoxy-5,5,6,6,6-pentafluoro-3-methylsulfonyl-1,4-dioxo-hex-2-en-2-olate Triethylammonium

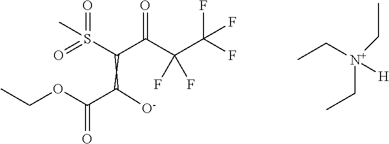

To a suspension of sodium 3-ethoxy-1-(methylsulfonyl)-3-oxoprop-1-en-2-olate (250 mg, 94% purity, 1.08 mmol) in acetonitrile (1.25 mL) was added triethylamine (1 mL, 7.17 mmol) that resulted to a thick suspension. The suspension was cooled to −10° C. and hexafluoropropylene oxide (25 mL, ca. 1.01 mmol) was added slowly via a syringe equipped with a rubber plunge. The solid sodium 3-ethoxy-1-(methylsulfonyl)-3-oxoprop-1-en-2-olate dissolved completely upon addition of the gas. The reaction mixture was then heated to room temperature and left at this temperature for 15 h. The reaction mixture was then evaporated under reduced pressure (16 mbar) leading to a red-brown oil (490 mg).

¹H NMR (DMSO-d₆, 600 MHz, 25° C.): δ (ppm)=8.88 (bs, 1H, ⁺HNEt₃), 4.07 (q, 2H, CH₃CH₂O, 7.2 Hz), 3.10 (q, 6H, CH₃CH₂N, 7.3 Hz), 2.93 (s, 3H, CH₃SO₂), 1.20 (t, 3H, CH₃CH₂O, 7.2 Hz), 1.17 (t, 9H, CH₃CH₂N, 7.3 Hz).

¹³C NMR (DMSO-d₆, 151 MHz, 25° C.): δ=179.6 (s), 175.3 (t, J_{C-F}=26.5 Hz), 166.0 (s), 119.1 (qt, CF₃, J_{C-F}=289.1; 35.6 Hz), 108.7 (tq, CF₂, J_{C-F}=271.0; 32.2 Hz), 108.5 (s), 60.1 (s), 45.8 (s), 43.2 (s), 13.8 (s), 8.6 (s).

¹⁹F NMR (DMSO-d₆, 377 MHz, 25° C.): δ (ppm)=−79.6 (s, 3F, CF₃), −117.0 (s, 2F, CF₂).

### Example 2

### Step (A)+(B)

### 2-Methyl-4-methylsulfonyl-5-(1,1,2,2,2-pentafluoroethyl)pyrazole-3-carboxylic Acid

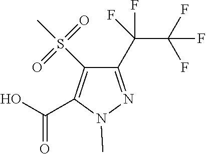 [SMILES: Cn1nc(C(F)(F)C(F)(F)F)c(S(C)(=O)=O)c1C(=O)O]

To a suspension of the sodium 3-ethoxy-1-(methylsulfonyl)-3-oxoprop-1-en-2-olate (1 g, 94% purity, 4.35 mmol) in tetrahydrofuran (5 mL) was added triethylamine (0.55 g, 5.44 mmol) that resulted to a thick suspension. Trifluoromethylbenzene (0.64 g, 4.34 mmol) was added to the reaction mixture as an internal standard to determine the yield in ¹⁹F NMR. Then hexafluoropropylene oxide (113 mL, ca. 4.57 mmol) was added slowly to the reaction mixture at 22° C. via a syringe equipped with a rubber plunge. The solid sodium 3-ethoxy-1-(methylsulfonyl)-3-oxoprop-1-en-2-olate dissolved completely upon addition of the gas. The reaction mixture was then stirred at the same temperature for 15 h. Then a solution of N-methylhydrazine in water (40%, 0.75 g, 6.52 mmol) was added to the reaction mixture and the resultant mixture was stirred at room temperature for 18 h. An aliquot of the reaction mixture (ca. 0.1 mL) was analyzed in ¹⁹F NMR (DMSO-d₆,) showing 77% yield. The reaction mixture was then evaporated under reduced pressure, the residue dissolved in water (3 mL) leading to a cloudy solution (pH 7). A solution of hydrochloric acid was then added (pH 1) resulting in a formation of a white precipitate. The precipitate was filtered off, washed with water (10 mL) and dried leading to 1.23 g of a white solid (88% purity, 77% yield).

¹H NMR (DMSO-d₆, 600 MHz, 25° C.): δ (ppm)=4.04 (s, 3H, CH₃N), 3.33 (s, 3H, CH₃SO₂), HO₂C exchanged with water peak.

¹³C NMR (DMSO-d₆, 151 MHz, 25° C.): δ=159.2 (s), 140.8 (s), 136.3 (t, J_{C-F}=30.9 Hz), 122.1 (s), 118.3 (qt, CF₃, J_{C-F}=286.7; 36.3 Hz), 109.8 (tq, CF₂, J_{C-F}=251.8; 38.2 Hz), 45.0 (s), 39.8 (s).

¹⁹F NMR (DMSO-d₆, 377 MHz, 25° C.): δ (ppm)=−81.2 (t, 3F, CF₃, J_{F-F}=2.1 Hz), −105.5 (q, 2F, CF₂, J_{F-F}=2.1 Hz).

### Example 3

### Step (A)+(B)

### 4-Methylsulfonyl-3-(1,1,2,2,2-pentafluoroethyl)-1H-pyrazole-5-carboxylic Acid

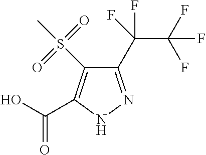 [SMILES: CS(=O)(=O)c1c(C(F)(F)C(F)(F)F)n[nH]c1C(=O)O]

To a suspension of sodium 3-ethoxy-1-(methylsulfonyl)-3-oxoprop-1-en-2-olate (1 g, 94% purity, 4.35 mmol) in tetrahydrofuran (5 mL) was added triethylamine (0.55 g, 5.44 mmol) that resulted to a thick suspension. Trifluoromethylbenzene (0.40 g, 2.74 mmol) was added to the reaction mixture as an internal standard to determine the yield in ¹⁹F NMR. Then hexafluoropropylene oxide (113 mL, ca. 4.57 mmol) was added slowly to the reaction mixture at 22° C. via a syringe equipped with a rubber plunge. The solid sodium 3-ethoxy-1-(methylsulfonyl)-3-oxoprop-1-en-2-olate dissolved completely upon addition of the gas. The reaction mixture was then stirred at the same temperature for 1 h. Then hydrazine hydrate (80%, 0.41 g, 6.52 mmol) was added to the reaction mixture and the resultant mixture was stirred at room temperature for 15 h. An aliquot of the reaction mixture (ca. 0.1 mL) was analyzed in ¹⁹F NMR (DMSO-d₆,) showing 89% yield. The reaction mixture was then evaporated under reduced pressure, the residue (ca. 2.55 g) dissolved in water (12 mL) leading to a cloudy solution. A solution was washed with ethyl acetate (10 mL). To the water phase a hydrochloric acid solution was then added (pH 1) resulting in a formation of a viscous oil. The mixture was extracted with ethyl acetate, the organic phase dried over Na₂SO₄, filtered and evaporated. The oily residue was recrystalized from ethyl acetate/n-heptane. The resultant precipitate was filtered off, washed with n-heptane and dried leading to 1.07 g of a yellowish solid (80% yield).

¹H NMR (DMSO-d₆, 600 MHz, 25° C.): δ (ppm)=15.5 (bs, 1H, HN), 3.39 (s, 3H, CH₃SO₂), HO₂C exchanged with water peak.

¹³C NMR (DMSO-d₆, 151 MHz, 25° C.): δ=158.6 (s), 139.1 (s), 138.5 (t, J_{C-F}=31.3 Hz), 123.0 (s), 118.6 (qt, CF₃, J_{C-F}=287.2; 35.9 Hz), 110.1 (tq, CF₂, J_{C-F}=251.4; 38.2 Hz), 44.7 (s).

¹⁹F NMR (DMSO-d₆, 377 MHz, 25° C.): δ (ppm)=−80.9 (s, 3F, CF₃), −104.7 (s, 2F, CF₂).

### Example 4

### Step (A)+(B)

### 2-Ethyl-4-methylsulfonyl-5-(1,1,2,2,2-pentafluoroethyl)pyrazole-3-carboxylic Acid

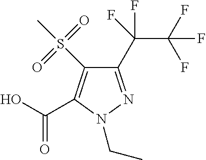 [SMILES: CCn1nc(C(F)(F)C(F)(F)F)c(S(C)(=O)=O)c1C(=O)O]

To a suspension of sodium 3-ethoxy-1-(methylsulfonyl)-3-oxoprop-1-en-2-olate (1 g, 94% purity, 4.35 mmol) in tetrahydrofuran (5 mL) was added triethylamine (0.55 g, 5.44 mmol) that resulted to a thick suspension. Trifluoromethylbenzene (0.40 g, 2.74 mmol) was added to the reaction mixture as an internal standard to determine the yield in ¹⁹F NMR. Then hexafluoropropylene oxide (113 mL, ca. 4.57 mmol) was added slowly to the reaction mixture at 22° C. via a syringe equipped with a rubber plunge. The solid sodium 3-ethoxy-1-(methylsulfonyl)-3-oxoprop-1-en-2-olate dissolved completely upon addition of the gas. The reaction mixture was then stirred at the same temperature for 1 h. Then N-ethylhydrazine (98%, 0.40 g, 6.52 mmol) was added to the reaction mixture and the resultant mixture was stirred at room temperature for 15 h. An aliquot of the reaction mixture (ca. 0.1 mL) was analyzed in ¹⁹F NMR (DMSO-d₆,) showing 84% yield. The reaction mixture was then evaporated at 25° C. under reduced pressure (40 mbar), the residue (ca. 2.6 g) dissolved in water (12 mL) leading to a cloudy solution. A solution was washed with ethyl acetate (10 mL). The organic phase was washed with water (2 mL). To the combined water fraction was added a solution of hydrochloric acid (pH 1) resulting in a formation of a precipitate. The precipitate was filtered off, washed with water and dried leading to 1.21 g of a white solid (83% yield).

¹H NMR (DMSO-d₆, 401 MHz, 25° C.): δ (ppm)=4.35 (q, 2H, CH₃CH₂O, 7.2 Hz), 3.32 (s, 3H, CH₃SO₂), 1.39 (t, 3H, CH₃CH₂O, 7.2 Hz), HO₂C exchanged with water peak.

¹³C NMR (DMSO-d₆, 151 MHz, 25° C.): δ=159.5 (s), 140.5 (s), 136.5 (t, J_{C-F}=30.9 Hz), 121.6 (s), 118.3 (qt, CF₃, J_{C-F}=286.8; 36.4 Hz), 109.9 (tq, CF₂, J_{C-F}=251.5; 38.7 Hz), 47.7 (s), 45.1 (s), 14.8 (s).

¹⁹F NMR (DMSO-d₆, 377 MHz, 25° C.): δ (ppm)=−81.3 (s, 3F, CF₃), −105.7 (s, 2F, CF₂).

### Example 5

### Step (A)+(B)

### 2-Isopropyl-4-methylsulfonyl-5-(1,1,2,2,2-pentafluoroethyl)pyrazole-3-carboxylic Acid

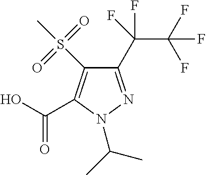 [SMILES: CC(C)n1nc(C(F)(F)C(F)(F)F)c(S(C)(=O)=O)c1C(=O)O]

To a suspension of sodium 3-ethoxy-1-(methylsulfonyl)-3-oxoprop-1-en-2-olate (500 mg, 94% purity, 2.17 mmol) in tetrahydrofuran (2.5 mL) was added triethylamine (275 mg, 2.72 mmol) that resulted to a thick suspension. Trifluoromethylbenzene (213 mg, 1.45 mmol) was added to the reaction mixture as an internal standard to determine the yield in ¹⁹F NMR. Then hexafluoropropylene oxide (56.5 mL, ca. 2.28 mmol) was added slowly to the reaction mixture at 22° C. via a syringe equipped with a rubber plunge. The solid sodium 3-ethoxy-1-(methylsulfonyl)-3-oxoprop-1-en-2-olate dissolved completely upon addition of the gas. The reaction mixture was then stirred at the same temperature for 1 h. Then N-isopropylhydrazine (95%, 254 mg, 3.26 mmol) was added to the reaction mixture and the resultant mixture was stirred at room temperature for 15 h. The reaction mixture was then evaporated under reduced pressure, the residue dissolved in water (20 mL) leading to a cloudy solution. A solution was washed with ethyl acetate (20 mL). To the water fraction was added a solution of hydrochloric acid (pH 1) resulting in a formation of a precipitate. The precipitate was filtered off, washed with water and dried leading to 621 mg of a white solid (82% yield).

¹H NMR (DMSO-d₆, 600 MHz, 25° C.): δ (ppm)=4.81 (hept, 1H, (CH₃)₂CHN, 6.5 Hz), 3.31 (s, 3H, CH₃SO₂), 1.43 (d, 6H, (CH₃)₂CHN, 6.5 Hz), HO₂C exchanged with water peak.

¹³C NMR (DMSO-d₆, 151 MHz, 25° C.): δ=159.7 (s), 140.4 (s), 136.4 (t, J_{C-F}=31.0 Hz), 120.9 (s), 118.3 (qt, CF₃, J_{C-F}=286.9; 36.4 Hz), 110.0 (tq, CF₂, J_{C-F}=251.9; 38.5 Hz), 54.6 (s), 45.2 (s), 22.0 (s).

¹⁹F NMR (DMSO-d₆, 377 MHz, 25° C.): δ (ppm)=−81.4 (t, 3F, CF₃, J_{F-F}=2.1 Hz), −105.8 (q, 2F, CF₂, J_{F-F}=2.1 Hz).

### Example 6

### Step (A)+(B)

### 2-Methyl-4-methylsulfonyl-5-(trifluoromethyl)pyrazole-3-carboxylic Acid

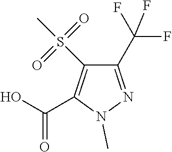 [SMILES: Cn1nc(C(F)(F)F)c(S(C)(=O)=O)c1C(=O)O]

To a suspension of the sodium 3-ethoxy-1-(methylsulfonyl)-3-oxoprop-1-en-2-olate (1 g, 94% purity, 4.35 mmol) in tetrahydrofuran (5 mL) was added triethylamine (1.0 g, 9.78 mmol) that resulted to a thick suspension. Trifluoromethylbenzene (0.41 g, 2.80 mmol) was added to the reaction mixture as an internal standard to determine the yield in ¹⁹F NMR. Then trifluoracetylchloride (113 mL, ca. 4.57 mmol) was added slowly to the reaction mixture at 22° C. via a syringe equipped with a rubber plunge. The solid sodium 3-ethoxy-1-(methylsulfonyl)-3-oxoprop-1-en-2-olate dissolved upon addition of the gas. The reaction mixture was then stirred at the same temperature for 30 min. Then a solution of N-methylhydrazine in water (40%, 0.75 g, 6.52 mmol) was added to the reaction mixture and the resultant mixture was stirred at room temperature for 3 days. An aliquot of the reaction mixture (ca. 0.1 mL) was analyzed in ¹⁹F NMR (DMSO-d₆,) showing 67% yield. The reaction mixture was then evaporated at 40° C. under reduced pressure (65 mbar), the residue dissolved in water leading to a cloudy solution. A solution was washed with ethyl acetate. To the water fraction was added a solution of hydrochloric acid (pH 1) resulting in a formation of an oil. The mixture was extracted with ethyl acetate, the organic phase dried over Na₂SO₄, filtered and evaporated. The oily residue was recrystalized from ethyl acetate/n-heptane. The resultant precipitate was filtered off, washed with n-heptane and dried leading to 0.69 g of a white solid (58% yield).

¹H NMR (DMSO-d₆, 600 MHz, 25° C.): δ (ppm)=4.05 (s, 3H, CH₃N), 3.34 (s, 3H, CH₃SO₂), HO₂C exchanged with water peak.

¹³C NMR (DMSO-d₆, 151 MHz, 25° C.): δ=159.1 (s), 140.4 (s), 137.6 (q, J_{C-F}=38.3 Hz), 120.9 (s), 119.7 (q, J_{C-F}=270.0 Hz), 44.8 (s), 39.9 (s).

¹⁹F NMR (DMSO-d₆, 377 MHz, 25° C.): δ (ppm)=−58.7 (s, CF₃).

### Example 7

### Step (A)+(B)

### 5-(1,1,2,2,3,3,3-Heptafluoropropyl)-2-methyl-4-methylsulfonyl-pyrazole-3-carboxylic Acid

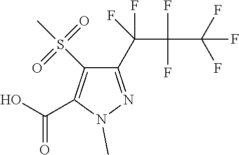 [SMILES: Cn1nc(C(F)(F)C(F)(F)C(F)(F)F)c(S(C)(=O)=O)c1C(=O)O]

To a suspension of the sodium 3-ethoxy-1-(methylsulfonyl)-3-oxoprop-1-en-2-olate (1 g, 95% purity, 4.39 mmol) in tetrahydrofuran (5 mL) was added triethylamine (0.56 g, 5.49 mmol) that resulted to a thick suspension. Trifluoromethylbenzene (0.43 g, 2.93 mmol) was added to the reaction mixture as an internal standard to determine the yield in ¹⁹F NMR. Then heptafluorobutyric anhydride (2 g, 4.79 mmol) was added slowly to the reaction mixture at 22° C. via a syringe. The solid sodium 3-ethoxy-1-(methylsulfonyl)-3-oxoprop-1-en-2-olate dissolved upon addition of the anhydride. The reaction mixture was then stirred at the same temperature for 1.5 h. Then a solution of N-methylhydrazine in water (40%, 0.76 g, 6.59 mmol) was added to the reaction mixture and the resultant mixture was stirred at room temperature for 18 h. An aliquot of the reaction mixture (ca. 0.1 mL) was analyzed in ¹⁹F NMR (DMSO-d₆,) showing 52% yield. The reaction mixture was then evaporated at 40° C. under reduced pressure, the residue dissolved in water leading to a cloudy solution. Then a water solution of hydrochloric acid was added leading to pH 1 and to precipitation of a sticky residue. The mixture was extracted with ethyl acetate, the organic phase dried over Na₂SO₄, filtered and evaporated. The oily residue was crystalized after addition of water. The resultant solid was filtered, washed with water and dried under vacuum leading to 0.78 g of a white solid (99.5% purity, 47% yield).

¹H NMR (DMSO-d₆, 401 MHz, 25° C.): δ (ppm)=5.32 (bs, HO₂C exchanged with water peak), 4.04 (s, 3H, CH₃N), 3.32 (s, 3H, CH₃SO₂).

¹³C NMR (DMSO-d₆, 101 MHz, 25° C.): δ=159.4 (s), 141.1 (s), 136.1 (t, J_{C-F}=30.0 Hz), 122.2 (s), 117.6 (qt, CF₃, J_{C-F}=288.8; 34.6 Hz), 112.0 (tt, CF₂, J_{C-F}=254.3; 32.4 Hz), 108.2 (ttq, CF₂, J_{C-F}=267.1; 37.6 Hz), 45.1 (s), 39.8 (s).

¹⁹F NMR (DMSO-d₆, 377 MHz, 25° C.): δ (ppm)=−79.5 (t, 3F, CF₃, J_{F-F}=10.0 Hz), −103.8 (q, 2F, CF₂, J_{F-F}=10.0 Hz), −123.6 (m, 2F, CF₂).

### Example 8

### Step (A)+(B)

### 4-(Benzenesulfonyl)-2-methyl-5-(1,1,2,2,2-pentafluoroethyl)pyrazole-3-carboxylic Acid

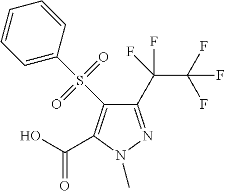 [SMILES: Cn1nc(C(F)(F)C(F)(F)F)c(S(=O)(=O)c2ccccc2)c1C(=O)O]

To a suspension of the sodium sodium 1-(benzenesulfonyl)-3-ethoxy-3-oxoprop-1-en-2-olate (1.4 g, 91% purity, 4.57 mmol) in tetrahydrofuran (7 mL) was added triethylamine (0.58 g, 5.72 mmol) that resulted to a thick suspension. Trifluoromethylbenzene (0.42 g, 2.83 mmol) was added to the reaction mixture as an internal standard to determine the yield in ¹⁹F NMR. Then hexafluoropropylene oxide (130 mL, ca. 5.26 mmol) was added slowly to the reaction mixture at 22° C. via a syringe equipped with a rubber plunge. The solid sodium 1-(benzenesulfonyl)-3-ethoxy-3-oxoprop-1-en-2-olate dissolved completely upon addition of the gas. The reaction mixture was then stirred at the same temperature for 1 h. Then a solution of N-methylhydrazine in water (40%, 0.79 g, 6.86 mmol) was added to the reaction mixture and the resultant mixture was stirred at room temperature for 18 h and then heated at 60° C. for 6 h. An aliquot of the reaction mixture (ca. 0.1 mL) was analyzed in ¹⁹F NMR (DMSO-d₆,) showing 80% yield. The reaction mixture was then evaporated under reduced pressure, the residue dissolved in water leading to a cloudy solution. A solution of hydrochloric acid was then added (pH 1) resulting in a formation of an oily residue. The mixture was extracted with ethyl acetate, the organic phase dried over Na₂SO₄, filtered and evaporated. The oily residue was recrystalized from ethyl acetate/n-heptane. The resultant precipitate was filtered off, washed with n-heptane and dried leading to to 1.31 g of a white solid (97.5% purity, 73% yield).

¹H NMR (DMSO-d₆, 401 MHz, 25° C.): δ (ppm)=8.11 (m, 2H), 7.68 (m, 1H), 7.61 (m, 2H), 4.61 (bs, HO₂C exchanged with water peak), 3.91 (s, 3H, CH₃N).

¹³C NMR (DMSO-d₆, 101 MHz, 25° C.): δ=159.6 (s), 146.1 (s), 141.6 (s), 135.7 (t, J_{C-F}=31.0 Hz), 133.7 (s), 129.2 (s), 127.5 (s), 118.7 (s), 118.2 (qt, CF₃, J_{C-F}=287.8; 36.7 Hz), 110.1 (tq, CF₂, J_{C-F}=251.0; 38.8 Hz), 38.9 (s).

¹⁹F NMR (DMSO-d₆, 377 MHz, 25° C.: δ (ppm)=−81.4 (s, 3F), −105.6 (s, 2F).

1. A process for preparation of one or more disubstituted 3-pyrazole carboxylates of formula (I) 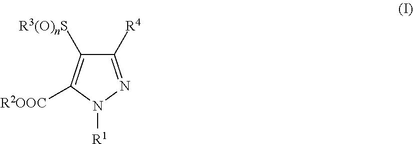
wherein
R¹ is selected from H, (C₁-C₆)alkyl, (C₃-C₈)cycloalkyl, phenyl or 2-pyridyl,
R² is selected from H, (C₁-C₁₂)alkyl or (C₃-C₈)cycloalkyl,
R³ is selected from (C₁-C₁₂)alkyl, (C₁-C₃)haloalkyl, (C₃-C₈)cycloalkyl, (C₆-C₁₂)aryl, (C₁-C₃)alkyl(C₆-C₁₂)aryl and (C₆-C₁₂)aryl(C₁-C₆)alkyl,
R⁴ is selected from (C₁-C₆)haloalkyl and (C₁-C₃)haloalkoxy(C₁-C₆)haloalkyl and
n is 0, 1 or 2,
comprising (A), wherein one or more acid derivatives of formula (II), 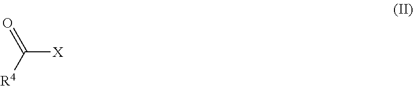
in which
R⁴ is defined as above and
X is selected from F, Cl, Br or —OC(O)R⁴
are, in the presence of a base, reacted with one or more enolates of formula (III), 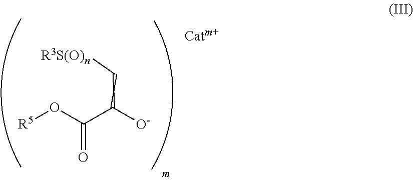
in which
R⁵ is selected from (C₁-C₁₂)alkyl, (C₆-C₁₂)aryl(C₁-C₆)alkyl, (C₆-C₁₂)aryl or (C₃-C₈)cycloalkyl,
n and R³ are defined as above,
m is 1 or 2 and
Cat^{m+} is selected from alkaline metal cations (with m=1), alkaline earth metal cations (with m=2), organic ammonium cations (with m=1) or organic phosphonium cations (with m=1)
to form one or more compounds of formula (IV) 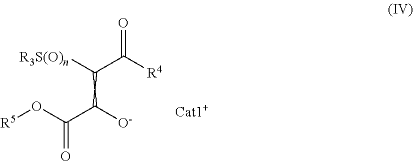
in which
n, R³, R⁴ and R⁵ are defined as above and
Cat1⁺ is selected from alkaline metal cations, N-methylimidazolium cation, N-butylimidazolium cation, pyridinium cation, (C₁-C₄)alkylpyridinium cations, dimethylaminopyridinium cation, 4-aza-1-azoniabicyclo[2.2.2]octane cation, 1-methyl-2,3,4,6,7,8,9,10-octahydropyrimido[1,2-a]azepin-1-ium cation or organic ammonium cations of the general formula (R⁶)₃NH⁺,
wherein
R⁶ are each independently selected from (C₁-C₆)alkyl or (C₃-C₈)cycloalkyl,
and further comprising (B), wherein cyclization with an hydrazine of formula (V)
NH₂NHR¹ (V)
takes place to form one or more of the compounds of formula (I).

2. A process according to claim 1, wherein the radicals in formula (I), (II), (III), (IV) and (V) are defined as
R¹ is selected from H, (C₁-C₆)alkyl, (C₃-C₈)cycloalkyl, phenyl or 2-pyridyl,
R² is selected from H, (C₁-C₆)alkyl or (C₃-C₆)cycloalkyl,
R³ is selected from (C₁-C₆)alkyl, (C₁-C₃)haloalkyl, (C₃-C₆)cycloalkyl, (C₆-C₉)aryl, (C₁-C₃)alkyl(C₆-C₉)aryl and (C₆-C₉)aryl(C₁-C₃)alkyl,
R⁴ is selected from (C₁-C₆)haloalkyl and (C₁-C₃)haloalkoxy(C₁-C₆)haloalkyl, wherein the halogen is selected from fluoro and/or chloro,
R⁵ is selected from (C₁-C₆)alkyl or (C₃-C₆)cycloalkyl,
n is 0, 1 or 2,
m is 1,
Cat^{m+} is selected from alkaline metal cations, preferably from Li⁺, Na⁺, K⁺ and Cs⁺, organic ammonium cations, preferably (R⁷)₄N⁺ or organic phosphonium cations, preferably (phenyl)₄P⁺, wherein
R⁷ are each independently selected from (C₁-C₆)alkyl or (C₆-C₁₂)aryl and
X is selected from F, Cl, Br or —OC(O)R⁴.

3. A process according to claim 1, wherein the radicals in formula (I), (II), (III), (IV) and (V) are defined as
R¹ is selected from H or (C₁-C₆)alkyl,
R² is selected from H or (C₁-C₆)alkyl,
R³ is selected from (C₆-C₉)aryl or (C₁-C₆)alkyl,
R⁴ is selected from difluoromethyl, trifluoromethyl, chlorofluoromethyl, dichlorofluoromethyl, chlorodifluoromethyl, 1-fluoroethyl, 2-fluoroethyl, 2,2-difluoroethyl, 2,2,2-trifluoroethyl, 2-chloro-2-fluoroethyl, 2-difluoroethyl, 2,2-dichloro-2-fluoroethyl, 2,2,2-trichloroethyl, 1,2,2,2-tetrafluoroethyl (CF₃CFH), pentafluoroethyl, heptafluoropropyl, trifluoromethoxyfluoromethyl(CF₃OCFH)— and 1,1,1-trifluoroprop-2-yl;
R⁵ is selected from (C₁₋₆)alkyl,
n is 0, 1 or 2,
m is 1,
Cat^{m+} is selected from Li⁺, Na⁺, K⁺ and Cs⁺ and (R⁷)₄N⁺, wherein
R⁷ are each independently selected from (C₁-C₂)alkyl and
X is independently selected from F, Cl, Br or —OC(O)R⁴.

4. A process according to claim 1, wherein the radicals in formula (I), (II), (III), (IV) and (V) are defined as
R¹ is selected from H, methyl, ethyl or iso-propyl,
R² is selected from H, methyl or ethyl,
R³ is selected from methyl, ethyl or phenyl,
R⁴ is selected from difluoromethyl, trifluoromethyl, pentafluoroethyl or heptafluoropropyl,
R⁵ is selected from methyl, ethyl, propyl or iso-propyl,
n is 2,
m is 1,
Cat^{m+} is selected from Li⁺, Na⁺, K⁺ and Cs⁺ and
X is F, Cl or —OC(O)R⁴.

5. A process according to claim 1, wherein the radicals in formula (I), (II), (III), (IV) and (V) are defined as
R¹ is selected from H or methyl,
R² is selected from H, methyl or ethyl,
R³ is methyl,
R⁴ is selected from trifluoromethyl, pentafluoroethyl or heptafluoropropyl,
R⁵ is selected from methyl or ethyl,
n is 2,
m is 1,
Cat^{m+} is selected from Na⁺ and K⁺ and
X is F, Cl or —OC(O)R⁴.

6. A process according to claim 1, wherein the base in (A) is selected from pyridine, (C₁-C₄)alkylpyridines, N-methylimidazole, N-butylimidazole, dimethylaminopyridine, 1,4-Diazabicyclo[2.2.2]octan (DABCO) and 1,8-diazabicyclo[5.4.0]undecene (DBU) or alkali metal hydroxides, alkali metal carbonates, alkali metal (C₁-C₄)alkoxides, alkali metal fluorides or trialkylamines (R⁶)₃N, wherein R⁶ are each independently selected from (C₁-C₆)alkyl or (C₃-C₈)cycloalkyl.

7. A process according to claim 6, wherein the base in (A) is selected from trialkylamines (R⁶)₃N, wherein R⁶ are each independently selected from (C₁-C₄)alkyl or (C₃-C₆)cycloalkyl.

8. A process according to claim 1, wherein the process is carried out in the presence of a suitable solvent and (B) is effected without changing the solvent after (A).

9. Intermediate of formula (IV) 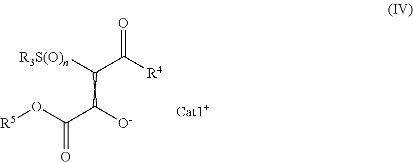
in which
R⁵ is selected from (C₁-C₁₂)alkyl, (C₆-C₁₂)aryl(C₁-C₆)alkyl, (C₆-C₁₂)aryl or (C₃-C₈)cycloalkyl,
R⁴ is selected from (C₁-C₆)haloalkyl and (C₁-C₃)haloalkoxy(C₁-C₆)haloalkyl and
n is 0, 1 or 2,
R³ is selected from (C₁-C₁₂)alkyl, (C₁-C₃)haloalkyl or (C₃-C₈)cycloalkyl and
Cat1⁺ is selected from alkaline metal cations, N-methylimidazolium cation, N-butylimidazolium cation, pyridinium cation, (C₁-C₄)alkylpyridinium cations, dimethylaminopyridinium cation, 4-aza-1-azoniabicyclo[2.2.2]octane cation, 1-methyl-2,3,4,6,7,8,9,10-octahydropyrimido[1,2-a]azepin-1-ium cation or organic ammonium cations of the general formula (R⁶)₃NH⁺,
wherein
R⁶ are each independently selected from (C₁-C₆)alkyl or (C₃-C₈)cycloalkyl.

10. Intermediate according to claim 9, wherein Cat1⁺ of formula (IV) is selected from organic ammonium cations of formula (R⁶)₃NH⁺,
wherein
R⁶ are each independently selected from (C₁-C₄)alkyl or (C₃-C₆)cycloalkyl.

11. Intermediate according to claim 9, wherein Cat1⁺ of formula (IV) is selected from N(iPr)₂(Et)H⁺, N(Me)₂(cyclohexyl)H⁺, N(Me)₃H⁺, N(Et)₃H⁺ or N(Bu)₃H⁺.

12. Intermediate according to claim 9, wherein
R³ is selected from (C₁-C₆)alkyl or (C₁-C₃)haloalkyl.

13. Disubstituted 3-pyrazole carboxylate of formula (I), 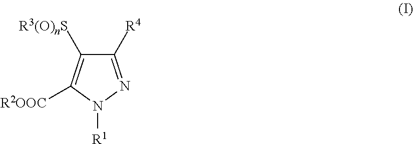
wherein
R¹ is selected from H, (C₁-C₆)alkyl, (C₃-C₈)cycloalkyl, phenyl or 2-pyridyl,
R² is selected from H, (C₁-C₁₂)alkyl or (C₃-C₈)cycloalkyl,
R³ is selected from (C₁-C₁₂)alkyl, (C₁-C₃)haloalkyl, (C₃-C₈)cycloalkyl, (C₆-C₁₂)aryl, (C₁-C₃)alkyl(C₆-C₁₂)aryl and (C₆-C₁₂)aryl(C₁-C₆)alkyl,
R⁴ is selected from (C₁-C₆)haloalkyl and (C₁-C₃)haloalkoxy(C₁-C₆)haloalkyl and
n is 2.

14. Disubstituted 3-pyrazole carboxylate according to claim 13, wherein
R¹ is selected from H, (C₁-C₆)alkyl, (C₃-C₈)cycloalkyl, phenyl or 2-pyridyl,
R² is selected from H, (C₁-C₆)alkyl or (C₃-C₆)cycloalkyl,
R³ is selected from (C₁-C₆)alkyl, (C₁-C₃)haloalkyl, (C₃-C₆)cycloalkyl, (C₆-C₉)aryl, (C₁-C₃)alkyl(C₆-C₉)aryl and (C₆-C₉)aryl(C₁-C₃)alkyl,
R⁴ is selected from (C₁-C₆)haloalkyl and (C₁-C₃)haloalkoxy(C₁-C₆)haloalkyl, wherein the halogen is selected from fluoro and/or chloro and
n is 2.

15. Disubstituted 3-pyrazole carboxylate according to claim 13, wherein
R¹ is selected from H or (C₁-C₆)alkyl,
R² is selected from H or (C₁-C₆)alkyl,
R³ is selected from (C₁-C₆)alkyl or (C₆-C₉)aryl,
R⁴ is selected from difluoromethyl, trifluoromethyl, chlorofluoromethyl, dichlorofluoromethyl, chlorodifluoromethyl, 1-fluoroethyl, 2-fluoroethyl, 2,2-difluoroethyl, 2,2,2-trifluoroethyl, 2-chloro-2-fluoroethyl, 2-difluoroethyl, 2,2-dichloro-2-fluoroethyl, 2,2,2-trichloroethyl, 1,2,2,2-tetrafluoroethyl (CF₃CFH), pentafluoroethyl, heptafluoropropyl, trifluoromethoxyfluoromethyl (CF₃OCFH)— and 1,1,1-trifluoroprop-2-yl and
n is 2.

16. Disubstituted 3-pyrazole carboxylate according to claim 13, wherein
R¹ is selected from H, methyl, ethyl or iso-propyl,
R² is selected from H, methyl or ethyl,
R³ is selected from methyl, ethyl or phenyl,
R⁴ is selected from difluoromethyl, trifluoromethyl, pentafluoroethyl or heptafluoropropyl and
n is 2.

17. Disubstituted 3-pyrazole carboxylate according to claim 13, wherein
R¹ is selected from H or methyl,
R² is selected from H, methyl or ethyl,
R³ is methyl,
R⁴ is selected from trifluoromethyl, pentafluoroethyl or heptafluoropropyl and
n is 2.
